(19) United States
(12) Patent Application Publication
(10) Pub. No.: US 2010/0223686 A1
(43) Pub. Date: Sep. 2, 2010

(54) MOUSE IN WHICH GENOME IS MODIFIED

(75) Inventor: Naoyuki TANIGUCHI, Suita-shi (JP)

Correspondence Address: SUGHRUE-265550, 2100 PENNSYLVANIA AVE. NW, WASHINGTON, DC, 20037-3213, US

(73) Assignee: KYOWA HAKKO KOGYO CO., LTD., Tokyo (JP)

(21) Appl. No.: 12/616,950

(22) Filed: Nov. 12, 2009

Related U.S. Application Data
(63) Continuation of application No. 12/261,997, filed on Oct. 30, 2008, now abandoned.
Application No. 12/261,997 is a continuation of application No. 11/783,487, filed on Apr. 10, 2007, now abandoned.
Application No. 11/783,487 is a continuation of application No. 10/803,100, filed on Mar. 18, 2004, now abandoned.
(60) Provisional application No. 60/501,019, filed on Sep. 9, 2003.

(30) Foreign Application Priority Data
Mar. 18, 2003 (JP) P. 2003-74195

(51) Int. Cl. A01K 67/027

(52) U.S. Cl. 800/18

(57) ABSTRACT

A mouse or progenies thereof in which genome is modified so as to have decreased or deleted activity of an enzyme relating to modification of a sugar chain in which the 1-position of fucose is bound to the 6-position of N-acetylglucosamine in the reducing end through α-bond in a complex N-glycoside-linked complex sugar chain.

The present application is a continuation of U.S. application Ser. No.. 12/,261,997, filed Oct. 30, 2008, which is a continuation of U.S. application Ser. No.. 11/783,487, filed Apr. 10, 2007 (abandoned), which is a continuation of U.S. application Ser. No.. 10/803,100, filed Mar. 18, 2004 (abandoned), which claims benefit of U.S. Provisional Application No. 60/501,019, filed Sep. 9, 2003 and JP 2003-74195, filed Mar. 18, 2003, the entire disclosures of each of which are hereby incorporated by reference.

### BACKGROUND OF THE INVENTION

1. Field of the Invention

The present invention relates to a mouse or progenies thereof in which genome is modified so as to have decreased or deleted activity of an enzyme relating to modification of a sugar chain in which the 1-position of fucose is bound to the 6position of N-acetylglucosamine in the reducing end through α-bond in a complex N-glycoside-linked complex sugar chain. 2. Brief Description of the Background Art

As the enzyme relating to modification of a sugar chain in which the 1-position of fucose is bound to the 6-position of N-acetylglucosamine in the reducing end through α-bond in a complex N-glycoside-linked complex sugar chain, α1,6-fucosyltransferase is known in the case of mammals (Biochem. Biophys. Res. Commun., 72, 909 (1976)). The structure of a gene encoding α1,6-fucosyltransferase (EC 2.4.1, 68) was found in 1996 (J. Biol. Chem., 271, 27817 (1996); J. Biochem., 121, 626 (1997); WO 92/27303). The enzyme activity of α1,6-fucosyltransferase has been found in many organs, and it has been reported that it is relatively high in the brain and small intestines (Int. J. Cancer, 72, 1117 (1997); Biochim. Biophys. Acta., 1473, 9 (1999)). Regarding its physiological functions, it has been pointed out that a fucose modified sugar chain plays an important role in the formation of retina, and attention has been paid to the relationship between retina formation and expression control of α1,6-fucosyltransferase (Glycobiology, 9, 1171 (1999). The role of human platelet-derived α1,6-fucosyltransferase in blood coagulation has also been pointed out (Biochem. Soc. Trans., 15, 603 (1987)). In addition, it has been also reported that modification of fucose to the sugar chain structure of immunoglobulin IgG1 changes binding of IgG1 to FcγRIIIa, and the antibody-dependent cellular cytotoxicity activity of the antibody itself is also changed (J. Biol. Chem., 277, 26733 (2002); J. Biol. Chem., 278, 3466 (2003)). Regarding its relation to morbid states of diseases, increase in the α1,6-fucosyltransferase activity and increase in the ratio of a reaction product of the enzyme have been observed in some diseases such as liver cancer and cystic fibrosis, so that its relation to these diseases has been pointed out (Hepatology, 13, 682 (1991), Hepatology, 28, 944. (1998)). It has been reported that an α1,6-fucosyltransferase hyperexpressing transgenic mouse was prepared, and an adiposis-like change was observed in the liver and kidney in the thus prepared transgenic mouse (Glycobiology, 11, 165 (2001)).

However, although WO 02/31140 discloses a transgenic nonhuman animal in which genome is modified so as to have decreased or deleted activity of an enzyme relating to modification of a sugar chain in which the 1-position of fucose is bound to the 6-position of N-acetylglucosamine in the reducing end through α-bond in a complex N-glycoside-linked complex sugar chain, there are no reports so far that a mouse in which genome is modified has been actually prepared.

### SUMMARY OF THE INVENTION

An object of the present invention is to provide a mouse or progenies thereof in which genome is modified so as to have decreased or deleted activity of an enzyme relating to modification of a sugar chain in which the 1-position of fucose is bound to the 6-position of N-acetylglucosamine in the reducing end through α-bond in a complex N-glycoside-linked complex sugar chain (hereinafter referred to as “α1,6-fucose modifying enzyme”).

The mouse and progenies thereof are useful in clarifying the physiological roes of the α1,6-fucose modifying enzyme and relation of the enzyme to morbid states of diseases. Furthermore, they are also useful in developing medicaments targeting at the α1,6-fucose modifying enzyme.

The present invention relates to the following (1) to (6).

(1) A mouse or progenies thereof in which genome is modified so as to have decreased or deleted activity of an enzyme relating to modification of a sugar chain in which the 1-position of fucose is bound to the 6-position of N-acetylglucosamine in the reducing end through α-bond in a complex N-glycoside-linked complex sugar chain.
(2) The mouse or progenies thereof according to (1), wherein a genomic gene of the enzyme relating to modification of a sugar chain in which the 1-position of fucose is bound to the 6-position of N-acetylglucosamine in the reducing end through α-bond in a complex N-glycoside-linked complex sugar chain is knocked out.
(3) The mouse or progenies thereof according to (1) or (2), wherein all alleles on the genome of the enzyme relating to modification of a sugar chain in which the 1-position of fucose is bound to the 6-position of N-acetylglucosamine in the reducing end through α-bond in a complex N-glycoside-linked complex sugar chain are knocked out.
(4) The mouse or progenies thereof according to any one of (1) to (3), wherein the enzyme relating to modification of a sugar chain in which the 1-position of fucose is bound to the 6-position of N-acetylglucosamine in the reducing end through α-bond in a complex N-glycoside-linked complex sugar chain is an α1,6-fucosyltransferase.
(5) The mouse or progenies thereof according to (4), wherein the α1,6-fucosyltransferase is a protein encoded by a DNA selected from the following (a) and (b):
(a) a DNA which comprises the nucleotide sequence represented by SEQ ID NO:2; and
(b) a DNA which hybridizes with the DNA comprising the nucleotide sequence represented by SEQ ID NO:2 under stringent conditions and encodes a protein having α1,6-fucosyltransferase activity.
(6) The mouse or progenies thereof according to (4), wherein the α1,6-fucosyltransferase is a protein selected from the group consisting of the following (a), (b) and (c):
(a) a protein which comprises the amino acid sequence represented by SEQ ID NO:1;
(b) a protein which comprises an amino acid sequence in which at least one amino acid in the amino acid sequence represented by SEQ ID NO:1 is deleted, substituted, inserted and/or added, and has α1,6-fucosyltransferase activity; and
(c) a protein which comprises an amino acid sequence having 80% or more of homology with the amino acid sequence represented by SEQ ID NO:1, and has α1,6-fucosyltransferase activity.

### BRIEF DESCRIPTION OF THE DRAWINGS

FIG. 1 is a graph showing a genomic region containing an exon positioned on the mouse FUT8 gene translation initiation codon.

FIG. 2 is a graph showing structure of a targeting vector for mouse FUT8 gene destruction use and its Southern blot judging method.

FIG. 3 is a graph showing Southern blotting of mouse genome in which PUTS allele was destructed.

FIG. 4 is a graph showing the Northern blotting which uses respective organs of a mouse in which FUT8 allele was destructed.

FIG. 5 is a graph showing α1,6-fucosyltransferase activity in a mouse in which FUT8 allele was destructed.

### DETAILED DESCRIPTION OF THE INVENTION

The mouse and progenies thereof of the present invention may be any mouse or progenies thereof, so long as they are a mouse or progenies thereof in which genome is modified so as to have decreased or deleted activity of the α1,6-fucose modifying enzyme.

In the present invention, the α1,6-fucose modifying enzyme includes any enzyme, so long as it is an enzyme relating to the reaction of binding of the 1-position of fucose to the 6-position of N-acetylglucosamine in the reducing end through α-bond in the complex N-glycoside-linked sugar chain. Specifically, the 1,6-fucose modifying enzyme includes α1,6-fucosyltransferase.

In the present invention, the α1,6-fucose modifying enzyme includes a protein encoded by a DNA of the following (a) or (b):

(a) a DNA comprising the nucleotide sequence represented by SEQ ID NO:2; and

(d) a DNA which hybridizes with the DNA comprising the nucleotide sequence represented by SEQ ID NO:2 under stringent conditions and encodes a protein having α1,6-fucosyltransferase activity; and

a protein of the following (c), (d) or (e)

(c) a protein comprising the amino acid sequence represented by SEQ ID NO:1;

(d) a protein which comprises an amino acid sequence in which at least one amino acid is deleted, substituted, inserted and/or added in the amino acid sequence represented by SEQ ID NO:1 and has α1,6-fucosyltransferase activity; and

(e) a protein which comprises an amino acid sequence having a homology of 80% or more with the amino acid sequence represented by SEQ ID NO:1 and has α1,6-fucosyltransferase activity.

In the present invention, a DNA which hybridizes under stringent conditions is a DNA obtained, e.g., by a method such as colony hybridization, plaque hybridization or Southern blotting hybridization which uses, as a probe, a DNA such as the DNA having the nucleotide sequence represented by SEQ ID NO:2 or a partial fragment thereof, and specifically includes a DNA which can be identified by carrying out hybridization at 65° C. in the presence of 0.7 to 1.0 M sodium chloride using a filter to which colony- or plaque-derived DNA fragments are immobilized, and then washing the filter at 65° C. using 0.1 to 2×SSC solution (composition of the 1×SSC solution comprising 150 mM sodium chloride and 15 mM sodium citrate). The hybridization can be carried out in accordance with the methods described, e.g., in Molecular Cloning, A Laboratory Manual, Second Edition, Cold Spring Harbor Laboratory Press (1989) (hereinafter referred to as “Molecular Cloning, Second Edition”), Current Protocols in Molecular Biology, John Wiley & Sons, 1987-1997 (hereinafter referred to as “Current Protocols in Molecular Biology”); DNA Cloning 1: Core Techniques, A Practical Approach, Second Edition, Oxford University (1995); and the like. The hybridizable DNA includes a DNA having a homology of at least 60% or more, preferably 70% or more, more preferably 80% or more, still more preferably 90% or more, far more preferably 95% or more, and most preferably 98% or more, with the nucleotide sequence represented by SEQ ID NO:2.

In the present invention, the protein which comprises an amino acid sequence in which at least one amino acid is deleted, substituted, inserted and/or added in the amino acid sequence represented by SEQ ID NO:1 and has α1,6-fucosyltransferase activity can be obtained, e.g., by introducing a site-directed mutation into a DNA encoding a protein having the amino acid sequence represented by SEQ ID NO:1 according to the site-directed mutagenesis described, e.g., in Molecular Cloning, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985); and the like. The number of amino acids to be deleted, substituted, inserted and/or added is one or more, and the number is not particularly limited, but is a number which can be deleted, substituted or added by a known technique such as the site-directed mutagenesis, e.g., it is 1 to several tens, preferably 1 to 20, more preferably 1 to 10, and most preferably 1 to 5.

Also, in the present invention, the protein which comprises an amino acid sequence having a homology of 80% or more with the amino acid sequence represented by SEQ ED NO:1 and has α1,6-fucosyltransferase activity is a protein having a homology of at least 80% or more, preferably 85% or more, more preferably 90% or more, still more preferably 95% or more, far more preferably 97% or more, and most preferably 99% or more, with the amino acid sequence represented by SEQ ID NO:1, when calculated by using an analyzing soft such as BLAST (J. Mol. Biol., 215, 403 (1990)), FASTA (Methods in Enzymology, 183, 63 (1990)) or the like.

In the present invention, modification of genome so as to have decreased or deleted activity of an α1,6-fucose modifying enzyme means that mutation is introduced into an expression-controlling region of the enzyme so as to decrease the expression of the enzyme, or that mutation is introduced into an amino acid sequence of the gene so as to decrease the function of the enzyme. Introduction of the mutation means that modification such as deletion, substitution, insertion and/or addition is carried out in the nucleotide sequence of the genome. Complete inhibition of the expression or function of the modified genomic gene is called “knock out”. The cell in which genomic gene is knocked out includes a cell in which a target gene is completely or partly deleted from the genome. As a method for obtaining such a mouse or progenies thereof, any technique can be used, so long as the genome of interest can be modified. Examples include a gene disruption technique which targets at a gene encoding the enzyme, a method for introducing mutation into a gene encoding the enzyme, a method for preparing a clone individual using the cell nucleus in which a gene of interest is modified, and the like.

Methods for preparing the mouse and progenies thereof of the present invention and methods for using them are described below in detail.

1. Method for Preparing the Mouse and Progenies thereof of the Present Invention
(1) Gene Disruption Technique which Targets at a Gene Encoding Enzyme

The mouse and progenies thereof of the present invention can be prepared by using a gene disruption technique which targets at a gene encoding the 1,6-fucose modifying enzyme. Specifically, the α1,6-fucose modifying enzyme includes α1,6-fucosyltransferase.

The gene disruption method may be any method, so long as it can disrupt the gene of the target enzyme. Examples include a homologous recombination method, an RDO method, a method using retrovirus, a method using transposon, and the like. The methods are specifically described below.

(a) Preparation of the Mouse and Progenies thereof of the Present Invention by Homologous Recombination

The mouse and the progenies thereof of the present invention can be produced by modifying a target gene on chromosome through a homologous recombination technique which targets at a gene encoding the α1,6-fucose modifying enzyme.

The target gene on chromosome can be modified by using a method described in Manipulating the Mouse Embryo, A Laboratory Manual, Second Edition, Cold Spring Harbor Laboratory Press (1994) (hereinafter referred to as “Manipulating the Mouse Embryo, A Laboratory Manual”); Gene Targeting, A Practical Approach, IRL Press at Oxford University Press (1993) (hereinafter referred to as “Gene Targeting, A Practical Approach”); Biomanual Series 8, Gene Targeting, Preparation of Mutant Mice using ES Cells, Yodo-sha (1995) (hereinafter referred to as “Preparation of Mutant Mice using ES Cells”); or the like, for example, as follows.

A cDNA encoding the α1,6-fucose modifying enzyme is prepared.

Based on the nucleotide sequence of the obtained cDNA, a genomic DNA encoding the α1,6-fucose modifying enzyme is prepared.

Based on the nucleotide sequence of the genomic DNA, a target vector is prepared for homologous recombination of a target gene to be modified (e.g., structural gene of the α1,6-fucose modifying enzyme, or a promoter gene).

The prepared target vector is introduced into an embryonic stem cell and a cell in which homologous recombination occurred between the target gene and target vector is selected.

The selected embryonic stem cell is introduced into a fertilized egg according to a known injection chimera method or aggregation chimera method, and the embryonic stem cell-introduced fertilized egg is transplanted into an oviduct or uterus of a pseudopregnant female mouse to thereby select germ line chimeras.

The selected germ line chimeras are crossed, and individuals having a chromosome into which the introduced target vector is integrated by homologous recombination with a gene region on the genome which encodes the α1,6-fucose modification enzyme are selected from the born offsprings.

The selected individuals are crossed, and homozygotes having a chromosome into which the introduced target vector is integrated by homologous recombination with a gene region on the genome which encodes the α1,6-fucose modification enzyme in both homologous chromosomes are selected from the born offsprings.

The obtained homozygotes are crossed to obtain offsprings to thereby prepare the mouse and progenies thereof of the present invention.

The method for obtaining a cDNA or a genomic DNA encoding the α1,6-fucosyltransferase includes the method described below.

Preparation Method of cDNA:

A total RNA or mRNA is prepared from mouse cells to be modified.

A cDNA library is prepared from the prepared total RNA or mRNA.

Degenerative primers are prepared based on known amino acid sequences encoding the α1,6-fucose modifying enzyme, e.g., human amino acid sequence, and a gene fragment encoding the α1,6-fucose modifying enzyme is obtained by CR method using the prepared cDNA library as the template.

A cDNA encoding the α1,6-fucose modifying enzyme can be obtained by screening the cDNA library by using the obtained gene fragment as a probe.

As the mRNA of mouse cells, a commercially available product (e.g., manufactured by Clontech) can be used, or the mRNA can be prepared from a total RNA prepared as follows. The method for preparing a total RNA from the cells includes the guanidine thiocyanate-cesium trifluoroacetate method (Methods in Enzymology, 154, 3 (1987)), the acidic guanidine thiocyanate phenol chloroform (AGPC) method (Analytical Biochemistry, 162, 156 (1987); Experimental Medicine (Jikken Igaku), 9, 1937 (1991)) and the like.

Furthermore, the method for preparing mRNA as poly(A)⁺ RNA from a total RNA includes the oligo(dT)-immobilized cellulose column method (Molecular Cloning, Second Edition) and the like.

In addition, mRNA can be prepared by using a kit such as Fast Track mRNA Isolation Kit (manufactured by Invitrogen), Quick Prep mRNA Purification Kit (manufactured by Pharmacia) or the like.

A cDNA library is prepared from the prepared mRNA of mouse cells. The method for preparing the cDNA library includes methods described in Molecular Cloning, Second Edition; Current Protocols in Molecular Biology; and the like; methods using a commercially available kits such as SuperScript Plasmid System for cDNA Synthesis and Plasmid Cloning (manufactured by Life Technologies) or ZAP-cDNA Synthesis Kit (manufactured by STRATAGENE); and the like.

As the cloning vector for the preparation of the cDNA library, any vector such as a phage vector, a plasmid vector or the like can be used, so long as it is autonomously replicable in Escherichia coli K12. Examples include ZAP Express (manufactured by STRATAGENE, Strategies, 5, 58 (1992)), pBluescript II SK(+) (Nucleic Acids Research, 17, 9494 (1989)), Lambda ZAP II (manufactured by STRATAGENE), λgt10 and λgt11 (DNA Cloning, A Practical Approach, 1, 49 (1985)), λTriplEx (manufactured by Clontech), λExCell (manufactured by Pharmacia), pcD2 (Mol. Cell. Biol., 3, 280 (1983)), pUC18 (Gene, 33, 103 (1985)) and the like.

Any microorganism can be used as the host microorganism, and Escherichia coli is preferably used. Examples include Escherichia coli MRF′ (manufactured by STRATAGENE, Strategies, 5, 81 (1992)), Escherichia coli C600 (Genetics, 39, 440 (1954)), Escherichia coli Y1088 (Science, 222, 778 (1983)), Escherichia coli Y1090 (Science, 222, 778 (1983)), Escherichia coli NM522 (J. Mol. Biol., 166, 1 (1983)), Escherichia coli K802 (J. Mol. Biol., 16, 118 (1966)), Escherichia coli JM105 (Gene, 38, 275 (1985)) and the like.

The cDNA library can be used as such in the subsequent analysis, and in order to obtain a full length cDNA as efficient as possible by decreasing the ratio of an infull length cDNA, a cDNA library prepared by using the oligo cap method developed by Sugano et al. (Gene, 138, 171 (1994); Gene, 200, 149 (1997); Protein, Nucleic Acid, Protein, 41, 603 (1996); Experimental Medicine (Jikken Igaku), 11, 2491 (1993); cDNA Cloning (Yodo-sha) (1996); Methods for Preparing Gene Libraries (Yodo-sha) (1994)) can be used in the following analysis.

Based on the amino acid sequence of the α1,6-fucose modifying enzyme, degenerative primers specific for the 5′-terminal and 3′-terminal nucleotide sequences of a nucleotide sequence presumed to encode the amino acid sequence are prepared, and DNA is amplified by PCR (PCR Protocols, Academic Press (1990)) using the prepared cDNA library as the template to obtain a gene fragment encoding the α1,6-fucose modifying enzyme.

It can be confirmed that the obtained gene fragment is a DNA encoding the α1,6-fucose modifying enzyme by a method generally used for analyzing a nucleotide, such as the dideoxy method of Sanger et al. (Proc. Natl. Acad. Sci. USA, 74, 5463 (1977)), a nucleotide sequence analyzer such as ABIPRISM 377 DNA Sequencer (manufactured by PE Biosystems) or the like.

A DNA encoding the α1,6-fucose modifying enzyme can be obtained by carrying out colony hybridization or plaque hybridization (Molecular Cloning, Second Edition) for the cDNA or cDNA library synthesized from the mRNA contained in the mouse cells to be modified, by using the gene fragment as a DNA probe.

Also, a DNA encoding the α1,6-fucose modifying enzyme can also be obtained by carrying out screening by PCR using the cDNA or cDNA library synthesized from the mRNA contained in the mouse cells to be modified as the template and using the primers used for obtaining the gene fragment encoding the α1,6-fucose modifying enzyme.

The nucleotide sequence of the obtained DNA encoding the α1,6-fucose modifying enzyme is analyzed from its terminus and determined by a method generally used for analyzing a nucleotide, such as the dideoxy method of Sanger et al. (Proc. Natl. Acad. Sci. USA, 74, 5463 (1977)), a nucleotide sequence analyzer such as ABIPRISM 377 DNA Sequencer (manufactured by PE Biosystems) or the like.

A gene encoding the α1,6-fucose modifying enzyme can also be determined from genes in data bases by searching nucleotide sequence data bases such as GenBank, EMBL and DDBJ by using a homology retrieving program such as BLAST based on the determined cDNA nucleotide sequence.

The nucleotide sequence of the gene encoding the α1,6-fucose modifying enzyme includes the nucleotide sequence represented by SEQ ID NO:2.

The cDNA encoding the α1,6-fucose modifying enzyme can also be obtained by chemically synthesizing it with a DNA synthesizer such as DNA Synthesizer model 392 manufactured by Perkin Elmer by using the phosphoamidite method, based on the determined DNA nucleotide sequence.

As a method for preparing a genomic DNA encoding the α1,6-fucose modifying enzyme, the method described below is exemplified.

### Preparation Method of Genomic DNA:

The method for preparing genomic DNA includes known methods described in Molecular Cloning, Second Edition; Current Protocols in Molecular Biology; and the like. In addition, a genomic DNA encoding the α1,6-fucose modifying enzyme can also be isolated by using a kit such as Genome DNA Library Screening System (manufactured by Genome Systems), Universal GenomeWalker™ Kits (manufactured by CLONTECH) or the like.

The nucleotide sequence of the genomic DNA encoding the α1,6-fucose modifying enzyme obtained by the above method can be confirmed based on the fact that it contains the cDNA sequence encoding the α1,6-fucose modifying enzyme obtained by the above method.

The target vector used in the homologous recombination of the target gene can be prepared in accordance with a method described in Gene Targeting, A Practical Approach; Preparation of Mutant Mice using ES Cells, Yodo-sha (1995); or the like. The target vector can be used as any of a replacement type, an insertion type and a gene trap type.

As the method for introducing the target vector into the embryonic stem cell, any method can be used, so long as it can introduce DNA into an animal cell. Examples include electroporation (Cytotechnology, 3, 133 (1990)), the calcium phosphate method (Japanese Published Unexamined Patent Application No. 227075/90), the lipofection method (Proc. Natl. Acad. Sci. USA, 84, 7413 (1987)), the injection method (Manipulating the Mouse Embryo, A Laboratory Manual), a method using particle gun (gene gun) (Japanese Patent No. 2606856, Japanese Patent No. 2517813), the DEAE-dextran method (Biomanual Series 4-Gene Transfer and Expression Analysis (Yodo-sha), edited by Takashi Yokota and Kenichi Arai (1994)), the virus vector method (Manipulating Mouse Embryo, A Laboratory Manual) and the like.

The method for efficiently selecting a homologous recombinant includes a method such as the positive selection, promoter selection, negative selection or polyA selection described in Gene Targeting, A Practical Approach; Preparation of Mutant Mice using ES Cells; or the like. Specifically, in the case of using the target vector comprising hprt gene, it is introduced into the hprt gene-defected embryonic stem cell, the embryonic stem cell is cultured in a medium comprising aminopterin, hypoxanthine and thymidine, and positive selection which selects the homologous recombinant of the hprt gene can be carried out by selecting a homogenous recombinant containing an aminopterin-resistant clone. In the case of using the target vector comprising a neomycin-resistant gene, the vector-introduced embryonic stem cell is cultured in a medium comprising G418, and positive selection can be carried out by selecting a homogenous recombinant containing a neomycin-resistant gene. In the case of using the target vector comprising DT gene, the vector-introduced embryonic stem cell is cultured, and negative selection can be carried out by selecting the grown clone which is a DT gene-free homogenous recombinant (since the DT gene is expressed while integrated in the chromosome, the recombinants introduced into a chromosome at random other than the homogenous recombination cannot grow due to the toxicity of DT). The method for selecting the homogenous recombinant of interest among the selected clones include the Southern hybridization for genomic DNA (Molecular Cloning, Second Edition), PCR (PCR Protocols, Academic Press (1990)) and the like.

When the embryonic stem cell is introduced into a fertilized egg by using an aggregation chimera method, in general, a fertilized egg at the development stage before 8-cell stage is preferably used. When the embryonic stem cell is introduced into a fertilized egg by using an injection chimera method, in general, it is preferred that a fertilized egg at the development stage from 8-cell stage to blastocyst stage is preferably used.

When the fertilized egg is transplanted into a female mouse, it is preferred that a fertilized egg obtained from a pseudopregnant female mouse in which fertility is induced by mating with a male non-human mammal which is subjected to vasoligation is artificially transplanted or implanted. Although the psuedopregnant female mouse can be obtained by natural mating, the pseudopregnant female mouse in which fertility is induced can be obtained by mating with a male mouse after administration of a luteinizing hormone-releasing hormone (hereinafter referred to as “LHRH”) or its analogue thereof. The analogue of LHRH includes [3,5-Dil-Tyr5]-LHRH, [Gln8]-LHRH, [D-Ala6]-LHRH, des-Gly10-[D-His(Bzl)6]-LHRH ethylamide and the like.

(b) Preparation of the Mouse and Progenies thereof of the Present Invention by an RDO Method

The mouse and progenies thereof of the present invention can be prepared according to an RDO (RNA-DNA oligonucleotide) method by targeting at a gene encoding the α1,6-fucose modifying enzyme, for example, as follows.

As described above, a cDNA or genomic DNA encoding the α1,6-fucose modifying enzyme is prepared, and the nucleotide sequence of the prepared cDNA or genomic DNA is determined.

Based on the determined DNA sequence, an RDO construct of an appropriate length comprising a part of a translation region, a part of an untranslated region or a part of intron of the target gene, is designed and synthesized.

The synthesized RDO is introduced into an embryonic stem cell and an embryonic stem cell in which the target enzyme, i.e., α1,6-fucose modifying enzyme, is mutated is selected.

The selected embryonic stem cell is introduced into a fertilized egg according to an injection chimera method or an aggregation chimera method, and the embryonic stem cell-introduced fertilized egg is transplanted into an oviduct or uterus of a pseudopregnant female mouse to thereby obtain germ line chimeras.

The selected germ line chimeras are crossed, and individuals having a chromosome into which the introduced target vector is integrated by homologous recombination with a gene region on the genome which encodes the α1,6-fucose modification enzyme are selected from the born offsprings.

The selected individuals are crossed, and homozygotes having a chromosome into which the introduced target vector is integrated by homologous recombination with a gene region on the genome which encodes an enzyme relating to the α1,6-fucose modification enzyme in both homologous chromosomes are selected from the born offsprings.

The obtained homozygotes are crossed to obtain offsprings to thereby prepare the mouse and the progenies thereof of the present invention.

The introduction of RDO into an embryonic stem cell can be carried out by the introduction method of the target vector described in the above item 1.(1)(a).

The RDO can be prepared by a usual method or using a DNA synthesizer.

The method for selecting an embryonic stem cell in which the α1,6-fucose modifying enzyme is mutated by introducing the RDO into the embryonic stem cell includes methods for directly detecting mutations in chromosomal genes described in Molecular Cloning, Second Edition; Current Protocols in Molecular Biology; and the like.

The construct of the RDO can be designed in accordance with the methods described in Science, 273, 1386 (1996); Nature Medicine, 4, 285 (1998); Hepatology, 25, 1462 (1997); Gene Therapy, 5, 1960 (1999); J. Mol. Med., 75, 829 (1997); Proc. Natl. Acad. Sci. USA, 96, 8774 (1999); Proc. Natl. Acad. Sci. USA, 96, 8768 (1999); Nuc. Acids. Res., 27, 1323 (1999); Invest. Dematol., 111, 1172 (1998); Nature Biotech., 16, 1343 (1998); Nature Biotech., 18, 43 (2000); Nature Biotech., 18, 555 (2000); and the like.

(c) Preparation of the Mouse and Progenies thereof of the Present Invention by a Method using a Transposon

The mouse and progenies thereof of the present invention can be prepared by using a transposon system described in Nature Genet., 25, 35 (2000) or the like, and then by selecting a mutant of the α1,6-fucose modifying enzyme.

The transposon system is a system in which a mutation is induced by randomly inserting an exogenous gene into chromosome, wherein an exogenous gene interposed between transposons is generally used as a vector for inducing a mutation, and a transposase expression vector for randomly inserting the gene into chromosome is introduced into the cell at the same time.

Any transposase can be used, so long as it is suitable for the sequence of the transposon to be used.

As the exogenous gene, any gene can be used, so long as it can induce a mutation in the DNA of the cell.

The introduction of the gene into the cell can be carried out by the introduction method of the target vector described in the above item 1.(1)(a).

(2) Method for Introducing Mutation into the Enzyme

The mouse and progenies thereof of the present invention can be prepared by introducing a mutation into a gene encoding the α1,6-fucose modifying enzyme, and then by selecting a mouse of interest in which the enzyme is mutated.

Specifically, the method includes a method in which a mouse of interest in which the mutation occurred in the gene encoding the α1,6-fucose modifying enzyme is selected from mutants born from generative cells which are subjected to mutation-inducing treatment or spontaneously generated mutants.

The generative cell includes cells capable of forming an individual such as a sperm, an ovum or an embryonic stem cell.

As the mutation-inducing treatment, any treatment can be used, so long as it can induce a point mutation, a deletion or frame shift mutation in the DNA of the cell. Examples include treatment with ethyl nitrosourea, nitrosoguanidine, benzopyrene or an acridine pigment and treatment with radiation. Also, various alkylating agents and carcinogens can be used as mutagens. The method for allowing a mutagen to act upon cells includes methods described in Tissue Culture Techniques, 3rd edition (Asakura Shoten), edited by Japanese Tissue Culture Association (1996), Nature Genet., 24, 314 (2000) and the like.

The spontaneously generated mutant includes mutants which are spontaneously formed by continuing general breeding without applying special mutation-inducing treatment,

(3) Method for Preparing a Clone Individual using the Cell Nucleus in which a Gene of Interest is Modified

The mouse and progenies thereof of the present invention can be prepared by the preparation method of clone mouse described in the literature (T. Wakayama, et al, Nature, 394, 369 (1988); T. Wakayama, et al, Nature Genetics, 22, 127 (1999)), for example, as described below.

Mutation is introduced into a gene encoding the α1,6-fucose modifying enzyme on the chromosome of any cell of a mouse by using the method described in the above items 1.(1) and (2).

Next, the nucleus of the obtained cell is initialized (i.e., is returned to the state in which the generation of the cell is repeated again).

The nucleus of the initialized cell is injected to an enucleated unfertilized egg of a mouse to thereby start the generation.

The egg which starts the generation is artificially transplanted and embedded into a female mouse to thereby obtain heterozygotes in which mutation is introduced into a gene encoding the α1,6-fucose modifying enzyme.

The obtained heterozygotes are crossed to thereby obtain homozygotes.

The obtained homozygotes are crossed to obtain offsprings to thereby prepare the mouse and the progenies thereof of the present invention.

It is known that the method for initializing the nucleus of the cell is different depending on the kind of the non-human mammal. In the case of a mouse, it is preferred that the initialization is carried out by injecting an exogeneous gene-introduced cell nucleus into an enucleated unfertilized egg of a conspecific non-human mammal, followed by culturing for several hours, preferably about 1 to 6 hours.

Also, it is known that the method for starting the generation of the initialized nucleus in the enucleated unfertilized egg is different depending on the kind of the non-human mammal. In the case of a mouse, it is preferred that the generation is started by stimulating an unfertilized egg into which an exogeneous gene-introduced cell nucleus is injected with a substance which activates an ovum (e.g., strontium, etc.) and treating it with an inhibitor of cell division (e.g., cytochalasin, etc.) to thereby inhibit release of the second polar body.

The method for artificially transplanting and embedding the egg which starts the generation into a female mouse includes the method described in the above item 1.(1)(a) and the like.

2. Use of the Mouse and Progenies thereof of the Present Invention
(1) Analysis of the Physiological Function of α1,6-fucose Modifying Enzyme using the Mouse and Progenies thereof of the Present Invention

Since the genome in the mouse and progenies thereof of the present invention is modified so as to have decreased or deleted activity of the α1,6-fucose modifying enzyme, it is possible to examine 1) physiological role of this enzyme in the process of development, 2) physiological role of this enzyme during processes after the development and reaching the adult body and 3) physiological role of this enzyme in the adult body. Also, α1,6-fucosyltransferase is known as the α1,6-fucose modifying enzyme, and the presence of an isozyme having similar enzyme activity can be clarified at various organ levels. In addition, it is possible to observe physiological influences by a quantitative change of the α1,6-fucose modifying enzyme, by comparing a normal individual, a heterozygote and a homozygote.

(2) Method for the Pharmacological Evaluation of Substances using the Mouse and Progenies thereof of the Present Invention

The mouse and progenies thereof of the present invention is useful in the case of a disease having a probability of being related to the α1,6-fucose modifying enzyme, as a tool for clarifying causal relation to the disease and finding its symptomatic therapy or radical therapy.

Specifically, pharmacological evaluation of a substance to be tested can be carried out by administering the substance to be tested to the mouse and progenies thereof of the present invention and comparing its pharmacological activities with those in an un-administered animal, for example by measuring various physical parameters such as blood pressure, respiration rate and body weight of the animal, observing its appearance and behavior or carrying out pathological and histological examinations. Information on symptoms similar to the symptoms observed in human diseases is often important, so that important data for the development of therapeutic drugs can be obtained.

Also, pharmacological evaluation of a substance to be tested, such as on its efficacy for a disease and side effects in an animal in which the activity of the α1,6-fucose modifying enzyme is decreased or deleted, can be carried out by preparing a pathological model animal in which the disease is induced in the mouse and progenies thereof of the present invention, administering the substance to be tested to the pathological model animal, carrying out, for example, measurement of various physical parameters such as blood pressure, respiration rate and body weight of the pathological model animal, observation of its morbid state, appearance and behavior or its pathological and histological examinations, and comparing the results with those of the pathological model animal without being administered with the substance to be tested. In addition, a substance desirable as the therapeutic drug for the disease can be selected based on this evaluation.

The diseases to be induced in the mouse and progenies thereof of the present invention include cardiac diseases (e.g., acute heart failure, chronic heart failure, myocarditis, etc.), respiratory diseases, joint diseases (e.g., articular rheumatism, osteoarthritis, etc.), renal diseases (e.g., renal insufficiency, glomerular nephritis, IgA glomerulonephritis, etc.), arteriosclerosis, psoriasis, hyperlipemia, allergic diseases (e.g., asthma, allergic rhinitis, atopic dermatitis, etc.), bone diseases (e.g., osteoporosis, rickets, osteomalacia, hypocalcemia, etc.), blood diseases, cerebrovascular injury, traumatic brain disorder, infection, dementia, cancer, diabetes mellitus, hepatic diseases, skin diseases, nerve degeneration diseases, chronic inflammatory diseases and the like. The pathological model animal can be prepared by the methods described in, for example, Manual of Disease Model Mice (Molecular Medicine, 31 Supplement, Nakayama Shoten (1994)), Pathological Animal Models for Pharmacology, Illustrated (Nishimura Shoten (1984)), Arthritis Model Animals (Ishiyaku Shuppan (1985)), Model Animals for Nerve and Muscle Diseases (Ishiyaku Shuppan (1982)) and Active Oxygen and Morbid States, From Disease Model To Bed Side (Gakkai Shuppan Center (1992)).

(3) Method for the Pharmacological Evaluation of Substances using Cells Obtained from the Mouse and Progenies thereof of the Present Invention

Pharmacological effects of substances to be tested on various cells obtained from the mouse and progenies thereof of the present invention can be evaluated by allowing the cells to contact with the substances to be tested and examining pharmacological activities including various responses Of the cells, such as increase in the intracellular Ca²⁺ concentration, and morphological changes of the cells, by comparing with cells in the absence of the substances to be tested.

In addition, various kinds of cells can be obtained by inducing differentiation of an embryonic stem cell obtained from the mouse and progenies thereof of the present invention. The method for inducing differentiation include a method for inducing a teratoma as a mixture of various tissues by transplanting an embryonic stem cell under the skin of a conspecific animal (Manipulating the Mouse Embryo, A Laboratory Manual) and a method for inducing its differentiation into an endodermal cell, an ectodermal cell, a mesodermal cell, a blood cell, an endothelial call, a cartilage cell, a skeletal muscle cell, a smooth muscle cell, a heart muscle cell, a nerve cell, a glial cell, an epithelial cell, a melanocyte or a keratinocyte (Reprod. Fertil. Dev., 10, 31, 1998) by culturing in vitro the stem cell under appropriate conditions. Pharmacological effects of substances to be tested on these cells after differentiation can be evaluated by allowing the cells to contact with the substances to be tested and examining pharmacological actions including various responses of the cells, such as increase in the intracellular Ca²⁺ concentration, and morphological changes of the cells, by comparing with cells in the absence of the substances to be tested. By these methods, pharmacological evaluation for cells which are difficult to be excised from the living body of a human patient or cells which are present therein in a small number can be carried out.

(4) Preparation of a Transgenic Animal using an Embryonic Stem Cell, Egg, Sperm or Nucleus of the Mouse and Progenies thereof of the Present Invention

A transgenic mouse in which genome is modified so as to have decreased or deleted activity of α1,6-fucose modifying enzyme and another gene on the chromosome is modified can be obtained according to the method described in the above item 1. by using an embryonic stem cell, an egg, a sperm or a nucleus obtained from the mouse and progenies thereof of the present invention. Particularly, a knockout mouse in which a gene commonly known to cause a morbid state by destroying a function of the gene is deleted, for example, by using the homologous recombination method described in the above item 1 and a transgenic mouse in which a dominant negative gene capable of inhibiting a function of the above-described gene is introduced and expressed are useful as pathological model animals.

(5) Preparation of a Transgenic Animal by Crossing the Mouse or Progenies Thereof of the Present Invention with a Conspecific Animal of Different Line, and Use of the Prepared Transgenic Animal

A transgenic mouse in which genome is modified so as to have decreased or deleted activity of α1,6-fucose modifying enzyme, and which shows certain phenotypic systems (e.g., symptoms similar to human morbid states), can be obtained by crossing the mouse and progenies thereof of the present invention with a conspecific animal of different line (e.g., human disease model animal). When the mouse to be used in the crossing is a human disease model animal, a pathological model animal in which genome is modified so as to have decreased or deleted activity of α1,6-fucose modifying enzyme and which shows symptoms similar to human morbid states as a phenotypic system is obtained. As the pathological model animal, any pathological model animal can be used, regardless of the congenital and acquired diseases. For example, acquired pathological model animals can be prepared by the methods described in, for example, Manual of Disease Model Mice (Molecular Medicine, 31 Supplement, Nakayama Shoten (1994)), Pathological Animal Models for Pharmacology, Illustrated (Nishimura Shoten (1984)), Arthritis Model Animals (Ishiyaku Shuppan (1985)), Model Animals for Nerve and Muscle Diseases (Ishiyaku Shuppan (1982)) and Active Oxygen and Morbid States, from Disease Model to Bed Side (Gakkai Shuppan Center (1992)).

(6) Method for the Pharmacological Evaluation of a Substance using a Transgenic Animal

Pharmacological evaluation of a substance to be tested, such as on its efficacy for diseases and side effects, can be carried out by administering the substance to be tested to the transgenic pathological model animal obtained by the methods described in the above items (4) and (5), carrying out, for example, measurement of various physical parameters such as blood pressure, respiration rate and body weight of the pathological model animal, observation of its morbid state, appearance and behavior or its pathological and histological examinations, and comparing the results with those of the pathological model animal without being administered with the substance to be tested. In addition, a substance desirable as the therapeutic drug for the disease can be selected based on this evaluation.

The present invention is described based on Examples in detail, but Examples merely show simple illustration of the present invention and the scope of the present invention is not limited thereto.

### EXAMPLE 1

Preparation of a Transgenic Mouse in which Both Alleles for α1,6-fucosyltransferase are Deleted:

A mouse in which a genomic region of both alleles for α1,6-fucosyltransferase (hereinafter referred to as “FUT8”) containing the translation initiation codon was deleted was prepared as described below.

### 1. Isolation of a Genomic Region Containing a Mouse FUT8 Gene Translation Initiation Codon

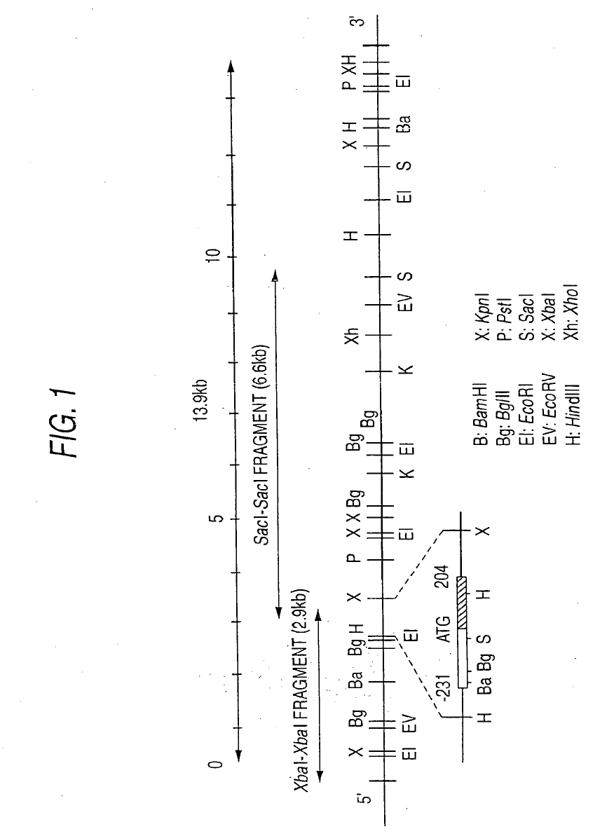

From the swine FUT8 full length cDNA (J. Biol. Chem., 271, 27810 (1996)), a fragment (373 bp) comprising 39th by non-translation region in the 5′-terminal side to 412th by translation region was prepared by digestion with a restriction enzyme SacI. Using this as the probe, a 13.9 Kb genomic clone comprising an exon which includes the mouse FUT8 translation initiation codon was isolated from a 129SVJ line mouse-derived λ-phage genomic library (manufactured by STRATAGENE) in accordance with the commonly known method described in Molecular Cloning, Second Edition (FIG. 1).

Next, after digestion of the thus obtained genomic clone using various restriction enzymes, Southern blotting was carried out in accordance with the commonly known method described in Molecular Cloning, Second Edition, by using the above-described swine FUT8 cDNA 412 by partial fragment as the probe. As a result, among the restriction enzyme fragments which showed positive reaction, an XbaI-XbaI fragment (about 2.9 Kb) encoding both the exon comprising the translation initiation codon and the upstream intron region and a SacI-SacI fragment (about 6.6 Kb) encoding both the exon comprising the translation initiation codon and the downstream intron region were selected and respectively inserted into pBluescript II KS(+) (manufactured by Stratagene) (FIG. 1).

### 2. Construction of a Targeting Vector Plasmid for Destruction of a Mouse FUT8 Gene Translation Initiation Codon

A targeting vector plasmid in which a SacI-HindIII region (184 bp) comprising the translation initiation codon was deleted was constructed by arranging an XbaI-SacI region (2.6 Kb) at the 5′-terminal side of the XbaI-XbaI region (about 2.9 Kb) obtained in the above item 1 of this Example as a 5′-terminal side homologous region, and a HindIII-XhoI region (about 6.1 Kb) at the 3′-terminal side of the SacI-SacI region (about 6.6 Kb) obtained in the item 1 of this Example as a 3′-terminal side homologous region. Its details are shown in the following.

First, a plasmid pMC1DTpA (Transgenic Research, 8, 215 (1999)) was digested with restriction enzymes XhoI and NotI, and a NotI-XhoI adapter was ligated to the thus obtained fragment of 1.5 Kb comprising a diphtheria toxin A chain (DT-A) gene, to thereby replace both termini with a XhoI recognizing region. On the other hand, the restriction enzyme XhoI was allowed to act upon pBluescript II KS(+) containing the SacI-SacI region (about 6.6 Kb) at the FUT8 3′-terminal side obtained in the above item 1 of this Example thereby obtain a fragment of 8.3 Kb containing the HindIII-XhoI region (about 6.1 Kb). By ligating the XhoI-XhoI fragment (8.3 Kb) containing the homologous region at the FUT8 3′-terminal side with the XhoI-XhoI fragment (1.5 Kb) containing the DT-A gene, both obtained in the above, to thereby construct a plasmid I.

Next, a fragment of 4.9 Kb comprising an expression unit for drug selection in which internal ribosome entry site (IRES), β-galactosidase gene (LacZ), neomycin resistant gene (Neo^{r}) and poly(A) addition signal (pA) were ligated (hereinafter referred to as “IRES-LacZ-Neo^{r}-pA cassette”) was obtained by completely digesting pGT1.8IresBgeo (Proc. Natl. Acad. Sci. U.S.A., 91, 4303 (1994)) with a restriction enzyme SalI and then partially digesting with a restriction enzyme SacI On the other hand, restriction enzymes SacI and SalI were allowed to act upon pBluescript II KS(+) containing the XbaI-XbaI region (about 2.9 Kb) at the FUT8 5′-terminal side obtained in the above item 1 of this Example to thereby obtain a fragment of 5.3 Kb containing the XbaI-SacI region (2.6 Kb). By ligating the SacI-SalI fragment (5.3 Kb) containing the homologous region at the FUT8 5′-terminal side with the SacI-SalI fragment (4.9 Kb) containing the IRES-LacZ-Neo^{r}-pA cassette, both obtained in the above, to thereby construct a plasmid II.

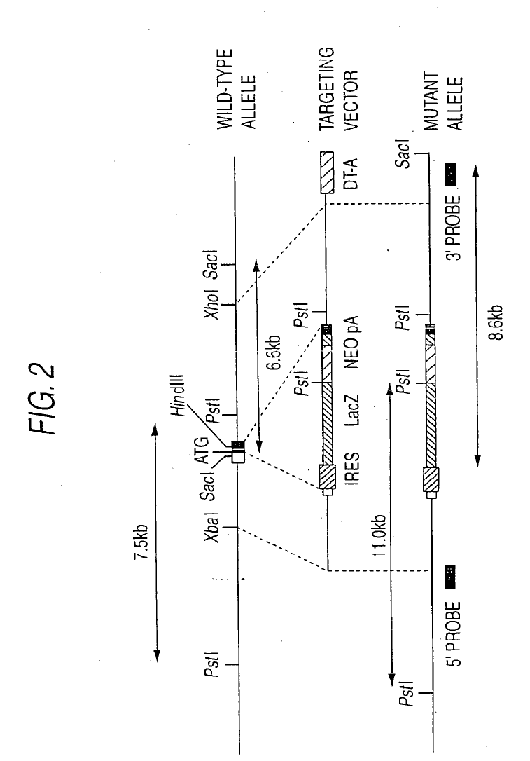

Finally, a targeting vector plasmid (17.0 Kb) for the destruction of mouse FUT8 gene was constructed by ligating a fragment of 9.4 Kb obtained by digesting the plasmid I with restriction enzymes NotI and SalI with a fragment of 7.6 Kb obtained by digesting the plasmid II with restriction enzymes NotI and SalI (FIG. 2).

### 3. Homologous Recombination of the Genomic Region of FUT8 Gene in a Mouse Embryonic Stem Cell

### (1) Preparation of Feeder Cells

The mouse embryonic stem cell used in the homologous recombination was cultured and maintained using mouse primary fibroblasts (EMFI cell) as the feeder. The feeder cells used for culturing and maintaining the embryonic stem cell were prepared in accordance with the following description.

First, in accordance with the description of Gene Targeting (Gene Targeting, A Practical Approach), a fetus of 13.5 days to 15.5 days after fertilization was excised from a neomycin resistant gene-introduced female mouse of 8 weeks or more old (received from Professor Masaru Okabe at the Laboratory for Genetic Information, Osaka University), a mouse primary fibroblast (EMFI cell) was prepared by using this as the material, and then its proliferation ability was inactivated by a mitomycin C treatment. Subsequently, the mitomycin-treated EMFI cells were suspended in an FM medium [Dulbecco's modified Eagle's medium (DMEM; manufactured by Invitrogen) supplemented with 10% fetal calf serum (manufactured by Invitrogen), 55 μmol/l β-mercaptoethanol (manufactured by Invitrogen), 1 mmol/l MEM sodium pyruvate (manufactured by Invitrogen), 0.1 mmol/l MEM nonessential amino acids (manufactured by Invitrogen), 3 mmol/l adenosine (manufactured by SIGMA), 3 mmol/l guanosine (manufactured by SIGMA), 3 mmol/l cytidine (manufactured by SIGMA), 3 mmol/l uridine (manufactured by SIGMA), 1 mmol/l thymidine (manufactured by SIGMA), 2 mmol/l L-glutamine (manufactured by Invitrogen), and 100 units/ml penicillin and 100 μg/ml streptomycin (manufactured by Invitrogen)] to a density of 2.5×10⁵ cells/ml, and then inoculated into a 0.1% gelatin coat-treated cell culture dish (6 cm in diameter or 10 cm in diameter; manufactured by Asahi Technoglass) or a 0.1% gelatin coat-treated flat bottom plate (96 wells, 24 wells or 6 wells; manufactured by Asahi Technoglass) and cultured at 37° C. for 24 hours in an atmosphere of 5% CO₂, thereby preparing a feeder plate. The thus prepared feeder plate was used within 1 week after its preparation. Also, in addition to the use of the above-described feeder cell, the Neo resistance primary culture cell manufactured by Life Tech Oriental (catalog number YE9284100) can also be used as the feeder cell.

(2) Introduction of a Targeting Vector into an Embryonic Stem Cell

Culturing of a mouse embryonic stem cell D3 (received from Professor Takashi Matsumura at the School of Medicine, Nagoya University, and preserved at the Laboratory for Genetic Information, Osaka University) was carried out by using an ESM medium [Dulbecco's modified Eagle's medium (DMEM; manufactured by Invitrogen) supplemented with 20% fetal calf serum (manufactured by Invitrogen), 55 μmol/l β-mercaptoethanol (manufactured by Invitrogen), 1 mmol/l MEM sodium pyruvate (manufactured by Invitrogen), 0.1 mmol/l MEM nonessential amino acids (manufactured by Invitrogen), 3 mmol/l adenosine (manufactured by SIGMA), 3 mmol/l guanosine (manufactured by SIGMA), 3 mmol/l cytidine (manufactured by SIGMA), 3 mmol/l uridine (manufactured by SIGMA), 1 mmol/l thymidine (manufactured by SIGMA), 2 mmol/l L-glutamine (manufactured by Invitrogen), 100 units/ml penicillin and 100 μg/m1 streptomycin (manufactured by Invitrogen), and 1,000 units/ml ESGRO™ (a mouse recombinant type leukemia inhibitory factor; manufactured by SIGMA Invitrogen)]. First, frozen D3p11 cell was thawed and inoculated into the feeder plate prepared by using a dish of 6 cm in diameter and cultured at 37° C. for 24 hours in an atmosphere of 5% CO₂. After the culturing, the D3 cells were sub-cultured in three feeder plates prepared by using dishes of 10 cm in diameter.

Gene transfer of the targeting vector plasmid obtained in the above item 2 of this Example into D3 cell was carried out in accordance with the electroporation (Cytotechnology, 3, 133 (1990)) as described below. First, 20 μg of the targeting vector plasmid was made into a linear form by digesting it with a restriction enzyme NotI, subjected to phenol/chloroform extraction treatment and ethanol precipitation and then made into a solution of 1 μg/μl. On the other hand, when 48 hours passed after sub-culturing of the D3p11 cells in the dish of 10 cm in diameter, the culture supernatant was discarded and replaced with fresh ESM medium. After confirming that the D3 cells reached 70% confluent, the cells were suspended in PBS buffer (manufactured by Invitrogen) to a density of 1×10⁷ cells/ml. After 800 μl (8.0×10⁶ cells) of the cell suspension was mixed with 20 μg of the above-described linearized plasmid, a total volume of the cell-DNA mixture was transferred into a Gene Pulser Cuvette (inter-electrode distance 4 mm) (manufactured by BIO-RAD) to carry out gene transfer by using a cell fusion apparatus Gene Pulser (manufactured by BIO-RAD) under conditions of 250 V in pulse voltage and 500 μF in electric capacity. After the gene transfer, the cell suspension was suspended in 40 ml of ESM medium and inoculated into 3 feeder plates prepared using dishes of 10 cm in diameter and 2 feeder plates prepared using dishes of 6 cm in diameter. After culturing for 16 hours or more under conditions of 5% CO₂ and 37° C., the culture supernatant was discarded and replaced with ESM medium supplemented with 150 μg/ml G418 (manufactured by Invitrogen). Drug-resistant strains were obtained by carrying out the culturing for 8 days while repeating this medium exchange procedure almost every day. Also, in addition to the above-described mouse embryonic stem cells, 129 line mouse-derived embryonic stem cells, such as mouse embryonic stem cell D3 (CRL-11632) which is available from ATCC or 129 line mouse-derived embryonic stem cells manufactured by Cell & Technologies or DNX Transgenic Sciences, USA, can also be used as the mouse embryonic stem cells.

(3) Preparation of a Targeting Vector-Transferred Strain

From the drug-resistant strains obtained in the above item (2), 343 optional colonies were collected as described below.

The culture supernatant was removed from the dish in which drug-resistant clones were formed and replaced with a phosphate buffer, and then the dish was placed under a stereoscopic microscope. Next, each colony was scraped out and sucked in using Pipette Man (manufactured by GILSON) and then transferred into a round bottom 96 well plate. After carrying out a trypsin treatment, each clone was inoculated into a feeder plate which had been prepared by using a flat bottom 24 well plate and cultured using ESM medium until it became confluent. After the culturing, each clone in the above plate was subjected to a trypsin treatment and its total volume was adjusted to 350 μl. After 300 μl thereof was mixed with the same volume of freezing medium (20% DMSO, 80% FM medium), the mixture was subjected to cryopreservation as a master plate. The remaining 50 μl of the cell suspension was inoculated into a gelatin coat-treated flat 24 well plate (manufactured by Asahi Technoglass) to be used as a replica plate and cultured by using ESM medium until the cells became confluent.

(4) Diagnosis of Homologous Recombination by Genomic Southern Blotting using a FUT8 Genomic Region 5′-Terminal Side Probe

With regard to the 343 clones obtained in the above item (3), diagnosis of homologous recombination was carried out by genomic Southern blotting using a 5′-terminal side probe, as described below.

First, genomic DNA of each clone was prepared from the replica plate prepared in the above item (3) in accordance with a known method (Nucleic Acids Research, 3, 2303 (1976)) and dissolved in a TE buffer (pH 8.0) (10 mmol/l Tris-HCl, 1 mmol/l EDTA).

On the other hand, an upstream fragment of about 500 bp from the restriction enzyme XbaI recognizing region at the 5′-terminal side was prepared from the genomic clone comprising the FUT8 translation initiation codon (13.9 Kb) obtained in the above item 1 of this Example (FIG. 2).

After digesting the genomic DNA with a restriction enzyme PstI, Southern blotting was carried out in accordance with the known method described in Molecular Cloning, Second Edition, by using the above-described FUT8 5′-terminal fragment (500 bp) as the probe.

By the treatment with a restriction enzyme PstI, a DNA fragment of about 7.5 Kb was formed from the wild type FUT8 allele. On the other hand, a DNA fragment of about 11.0 Kb was formed by the same restriction enzyme treatment from the allele in which homologous recombination with the targeting vector was generated (FIG. 2).

By this method, specific fragments of about 7.5 Kb and about 11.0 Kb were found in genomic DNA samples prepared from 4 clones. Since the quantitative ratio of both fragments was 1:1, it was confirmed that the clone is a homologous recombinant in which one of the FUT8 allele was substituted by a vector sequence.

(5) Diagnosis of Homologous Recombination by Genomic Southern Blotting using a FUT8 Genomic Region 3′-Terminal Side Probe

With regard to the 343 clones obtained in the above item (3), diagnosis of homologous recombination was carried out by genomic Southern blotting using a 3′-terminal side probe, as described below.

First, a fragment comprising a restriction enzyme EcoRV recognizing region, positioned at about 500 by upstream from the restriction enzyme SacI recognizing region at the 3′-terminal moiety, was prepared from the SacI-SacI region (about 6.3 Kb) obtained in the item 1 of this Example.

After digesting the genomic DNA prepared in the item (4) with the restriction enzyme SacI, Southern blotting was carried out in accordance with the known method described in Molecular Cloning, Second Edition, by using the above-described FUT8 3′-terminal fragment (500 bp) as the probe.

By the treatment with a restriction enzyme SacI, a DNA fragment of about 6.6 Kb was formed from the wild type FUT8 allele. On the other hand, a DNA fragment of about 8.6 Kb was formed by the same restriction enzyme treatment from the allele in which homologous recombination with the targeting vector was generated (FIG. 2).

By this method, specific fragments of about 6.6 Kb and about 8.6 Kb were found in genomic DNA samples of 4 clones which showed positive result in the above item (4). Since the quantitative ratio of both fragments was 1:1, it was confirmed that the clone is a homologous recombinant in which one of the FUT8 allele was substituted by a vector sequence.

4. Preparation of a Mouse in which an FUT8 Gene is Destroyed
(1) Preparation of a Chimeric Mouse by using an Embryonic Stem Cell in which 1 Copy of FUT8 Alleles is Destroyed

From the 4 embryonic stem cell clones established in the above item 3 of this Example in which one of the FUT8 alleles was destroyed, 3 clones keeping the normal karyotype were selected in accordance with a conventional method (Manipulating the Mouse Embryo, A Laboratory Manual). Next, in accordance with the injection chimera method described, for example, in Guide to Techniques in Mouse Development, Methods in Enzymology, Volume 225, Academic Press (1993), each of the 3 embryonic stem cell clones was injected under a microscopy into the cavity of blastocyst prepared from a C57BL/6 line female mouse and transplanted and embedded in the uterus of a pseudopregnant MCH line female mouse.

Among male chimeric individuals having brown hair showing up in the black hair, an individual having a chimeric ratio of exceeding 50% was judged that the injected embryonic stem cell is contributing to a germ cell line at an equivalent level so that it was subjected to crossing with a C57BL/6 line male mouse. As a result, it was confirmed that a chimera of germ line was present in chimeric individuals prepared by using 2 clones of the embryonic stem cells.

(2) Preparation of Heterozygote Mouse in which 1 Copy of FUT8 Allele is Destroyed

After rearing the germ line chimera obtained in the above item (1) until 8 weeks old, it was crossed with a sexually matured C57BL/6 line female individual to obtain offsprings. Among these offsprings, a genomic DNA was prepared from the tail of an individual having brown hair in accordance with a known method (Nucleic Acids Research, 3, 2303 (1976)), and Southern blotting was carried out in accordance with the method described in the above item 3(4) of this Example.

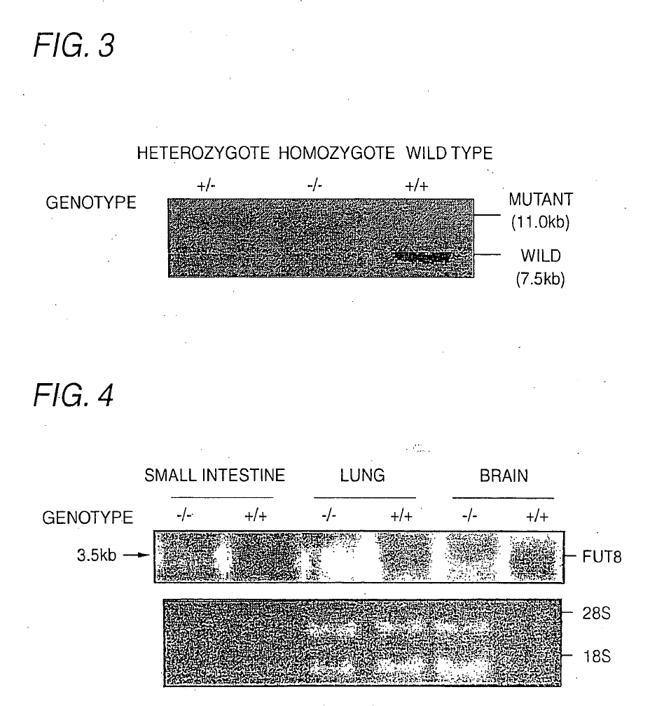

By its treatment with a restriction enzyme PstI, the heterozygote genomic DNA formed a wild type FUT8 allele-specific fragment of about 7.5 Kb and a homologous recombination-caused allele-specific fragment of about 11.0 Kb at a quantitative ratio of 1:1 (FIG. 2). As a result of the Southern blotting, it was confirmed that the heterozygote satisfying the above-described judging criteria was obtained from chimeric individuals derived from each of the 2 embryonic stem cell clones whose contribution to the germ line was found in the above item (1) (FIG. 3). This heterozygote mouse was a mouse in which one of the FUT8 allele was destroyed.

(3) Preparation of Homozygote Mouse in which FUT8 Alleles are Destroyed

After rearing the heterozygote male individual and female individual obtained in the above item (2) until 8 weeks old, they were crossed to obtain offsprings. Among these offsprings, genomic DNA of each clone was prepared from the tail of an individual having brown hair in accordance, with a known method (Nucleic Acids Research, 3, 2303 (1976)), and Southern blotting was carried out in accordance with the method described in the above item 3(4) of this Example.

By its treatment with a restriction enzyme PstI, the homozygote genomic DNA forms only a wild type FUT8 allele-specific fragment of about 7.5 Kb (FIG. 2). As a result of the Southern blotting, it was confirmed that the homozygote satisfying the above-described judging criteria was contained in the offsprings (FIG. 3). This homozygote mouse was a mouse in which both of the FUT8 alleles were destroyed.

### EXAMPLE 2

Expression Analysis of Both FUT8 Alleles in Transgenic Mouse in which the Alleles are Deleted:

### 1. Analysis of Expressed Amount of the Gene in FUT8 Double Knockout Mouse

Small intestines, lungs and brains were excised from the FUT8 knockout homozygote mouse prepared in the above item 4 of Example 1 and a wild type mouse, and the total RNA was prepared in accordance with a known method described, for example, in Molecular Cloning, Second Edition. After 1.0% (w/v) agarose gel electrophoresis containing 2.2 mol/l formaldehyde was carried out by using 20 μg of the total RNA obtained from each organ, the RNA was transferred onto Zeta-probe membrane (manufactured by BIO-RAD) in accordance with a known method (Proc. Natl. Acad. Sci. USA, 76, 3683 (1979)).

On the other hand, a probe was prepared by ³²P-labeling a human FUT8 complete length cDNA (J. Biochem., 121, 626 (1997)). Northern hybridization was carried out in accordance with the known method described in Molecular Cloning, Second Edition, by allowing the thus prepared probe and membrane to react at 55° C.

After the hybridization, the membrane was soaked in 2×SSC-0.1% (w/v) SDS and incubated at 55° C. for 30 minutes. After repeating the above washing procedure again, the washed membrane was exposed to an X-ray film at −80° C. for 3 days to develop images.

By this method, expression of the mouse FUT8 complete length mRNA of about 3.5 Kb can be detected. As a result of the Northern blotting, a single band of about 3.5 K was detected in all organs obtained from the wild type mouse. On the other hand, a band corresponding to the FUT8 mRNA was unable to be detected in the organs obtained from the FUT8 double knockout mouse (FIG. 4). Thus, it was confirmed that expression of the FUT8 mRNA was deleted in the FUT8 double knockout mouse.

2. Measurement of FUT8 Activity in FUT8 Double Knockout Mouse

Brains and small intestines were excised from the FUT8 knockout heterozygote mouse and homozygote mouse prepared in the above item 4 of Example 1 and a wild type mouse, and each organ was pulverized in 4 volumes of 0.25 mol/l sucrose-1.0 mol/l benzamidine-10 mmol/l Tris-HCl buffer (pH 7.4). After centrifugation at 900×g for 10 minutes, the thus recovered supernatant was used as a crude enzyme. The enzyme reaction was measured by incubating 35 μl of a reaction solution [50 mmol/l MES-NaOH buffer (pH 7.0), 0.3% Triton X-100, 0.285 mmol/l GDP-fucose, 4.2 μmol/l 4-(2-pyridylamino)butylamine-labeled (asparagine-linked) agalacto biantennary sugar chain (J. Biol. Chem., 271, 27810 (1996)), containing from. 30 μg to 90 μg of each crude enzyme, at 37° C. for 2 hours. The reaction was terminated by heating at 100° C. for 1 minute. Regarding the reaction using a crude enzyme prepared from the FUT8 knockout homozygote mouse, it was measured by incubating for 12 hours. After termination of the reaction with heating, 10 μl of a supernatant recovered by carrying out centrifugation at 5,000×g for 10 minutes was prepared as a sample. Next, each of the thus prepared samples was injected into a TSK-gel ODS-80TM column (4.6×150 mm, manufactured by Tosoh) attached to a LC-VP HPLC system (manufactured by Shimadzu) and developed by using 0.15% 1-butanol-20 mmol/l sodium acetate buffer (pH 4.0) at 55° C. and at a flow rate of 1.0 ml/min, and then fluorescence intensity (excitation wavelength: 320 nm, detection wavelength: 400 nm) of the eluted reaction product was measured.

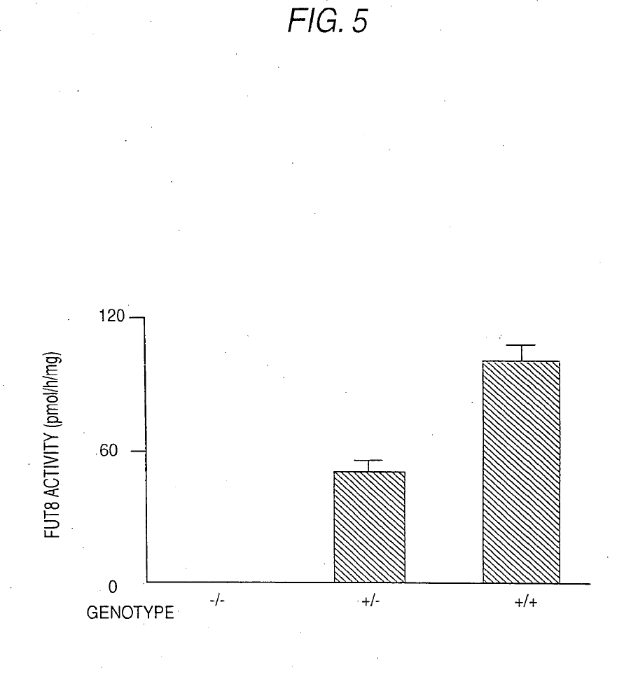

As a result of this enzyme activity measurement, the FUT8 activity was not detected in the small intestines of the FUT8 knockout homozygote mouse, even when the reaction period was prolonged to 6 times than that of the case of wild type mouse-derived organs. On the other hand, the FUT8 activity was detected in response to the expressed amount of the FUT8 gene, in the brains of the FUT8 knockout heterozygote mouse and wild type mouse (FIG. 5). Thus, it was confirmed that the FUT8 activity was deleted in the FUT8 double knockout mouse.

While the invention has been described in detail and with reference to specific embodiments thereof, it will be apparent to one skill in the art that various changes and modifications can be made therein without departing from the spirit and scope thereof. All references cited herein are incorporated in their entirety.

This application is based on Japanese patent application No. 2003-074195 filed on Mar. 18, 2003 and U.S. provisional patent application No. 60/501,019 filed on Sep. 9, 2003, the entire contents of which being incorporated hereinto by reference.

What is claimed is:

1. A mouse, or progenies thereof, in which the genome is modified such that said mouse, or progenies thereof, have decreased or deleted α1,6-fucosyltransferase activity.

2. The mouse, or progenies thereof, according to claim 1, wherein the genomic gene encoding α1,6-fucosyltransferase is knocked out.

3. The mouse, or progenies thereof, according to claim 1, wherein both α1,6-fucosyltransferase alleles in the genome of said mouse, or progenies thereof, are knocked out.

4. The mouse, or progenies thereof, according to claim 1, wherein the α1,6-fucosyltransferase is a protein encoded by a DNA which comprises the nucleotide sequence as set forth in SEQ ID NO:2.

5. The mouse, or progenies thereof, according to claim 1, wherein the α1,6-fucosyltransferase is a protein selected from the group consisting of the following (a) and (b):
(a) a protein which comprises the amino acid sequence as set forth in SEQ ID NO:1; and
(b) a protein which comprises an amino acid sequence having 80% or more homology with the amino acid sequence set forth in SEQ ID NO:1.


SEQUENCE LISTING (2 sequences)

SEQ ID NO: 1 - protein, 575 aa, Mus musculus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 2 - DNA, 1728 bp, Mus musculus
atgcgggcatggactggttcctggcgttggattatgctcattctttttgcctgggggaccttgttattttatataggtggtcatttggttcgagataatgaccaccctgatcactccagcagagaactctccaagattcttgcaaagcttgaacgcttaaaacagcaaaatgaagacttgaggcgaatggctgagtctctccgaataccagaaggccccattgaccaggggacagctacaggaagagtccgtgttttagaagaacagcttgttaaggccaaagaacagattgaaaattacaagaaacaagctagaaatggtctggggaaggatcatgaaatcttaagaaggaggattgaaaatggagctaaagagctctggttttttctacaaagcgaactgaagaaattaaagcatttagaaggaaatgaactccaaagacatgcagatgaaattcttttggatttaggacaccatgaaaggtctatcatgacagatctatactacctcagtcaaacagatggagcaggggattggcgtgaaaaagaggccaaagatctgacagagctggtccagcggagaataacatatctccagaatcctaaggactgcagcaaagccaggaagctggtgtgtaacatcaataaaggctgtggctatggttgtcaactccatcacgtggtctactgtttcatgattgcttatggcacccagcgaacactcatcttggaatctcagaattggcgctatgctactggtggatgggagactgtgtttagacctgtaagtgagacatgtacagacagatctggcctctccactggacactggtcaggtgaagtaaatgacaaaaacattcaagtggtcgagctccccattgtagacagcctccatcctcggcctccttacttaccactggctgttccagaagaccttgcagaccgactcctaagagtccatggtgaccctgcagtgtggtgggtgtcccagtttgtcaaatacttgattcgtccacaaccttggctggaaaaggaaatagaagaagccaccaagaagcttggcttcaaacatccagttattggagtccatgtcagacgcacagacaaagtgggaacagaagcagccttccaccccatcgaggagtacatggtacacgttgaagaacattttcagcttctcgcacgcagaatgcaagtggataaaaaaagagtatatctggctactgatgatcctactttgttaaaggaggcaaagacaaagtactccaattatgaatttattagtgataactctatttcttggtcagctggactacacaatcggtacacagaaaattcacttcggggtgtgatcctggatatacactttctctcacaggctgactttctagtgtgtactttttcatcccaggtctgtcgggttgcttatgaaatcatgcaaaccctgcatcctgatgcctctgcgaacttccattctttggatgacatctactattttggaggccaaaatgcccacaatcagattgctgtttatcctcacaaacctcgaactgaagaggaaattccaatggaacctggagatatcattggtgtggctggaaaccattgggatggttattctaaaggtatcaacagaaaacttggaaaaacaggcttatatccctcctacaaagtccgagagaagatagaaacagtcaagtatcccacatatcctgaagctgaaaaatag